(19) United States
(12) Patent Application Publication
(10) Pub. No.: US 2016/0158265 A1
(43) Pub. Date: Jun. 9, 2016

(54) ISOLATION OF NOVEL BIOACTIVE COMPOUND OBTAINED FROM OIL PALM BASE MATERIALS

(71) Applicant: MALAYSIAN PALM OIL BOARD (MPOB), Selangor (MY)

(72) Inventors: Ravigadevi SAMBANTHAMURTHI, Selangor (MY); Yew Ai TAN, Selangor (MY); Saridah Wan Omar WAN, Selangor (MY); Md Ali JABARIAH, Selangor (MY); Sambandan TG, Selangor (MY); Meng Fei YANG, Selangor (MY); CHOKYUN Rha, Selangor (MY); Anthony J. SINSKEY, Selangor (MY)

(73) Assignee: MALAYSIAN PALM OIL BOARD, Selangor (MY)

(21) Appl. No.: 14/900,965

(22) PCT Filed: Jun. 17, 2014
(86) PCT No.: PCT/MY2014/000184

§ 371 Date: Dec. 22, 2015

(30) Foreign Application Priority Data
Jun. 28, 2013 (MY) PI2013002518

(51) Int. Cl. A61K 31/7056; A61K 36/889

(52) U.S. Cl. CPC A61K 31/7056; A61K 36/889; A61K 2236/00

(57) ABSTRACT

Present invention relates to a composition comprising novel bioactive compound obtained from oil palm based materials and the method of isolating bioactive compound thereof, wherein said compound is an indolacetic acid derivative. The compound is having at least one variation of moiety.

### FIELD OF INVENTION

The invention generally relates to bioactive compounds and more particularly isolating, purifying and characterizing a phenolic compound obtained from plants and plant-based material, with said compound exhibiting highly significant bioactive properties.

### BACKGROUND OF INVENTION

Bioactive compounds are essential due to its importance to in interacting With or effect on any cell tissue in the human body, as well as to provide beneficial pharmacological activity in treating diseases. In view of various disease outbreaks and sky-rocketing mortality rate, the demands in such bioactive compounds are constantly increasing. Therefore, it is an object of present invention to provide plant-based compound with bioactive properties.

Present invention focuses on realizing the value and potential of the vegetation liquor and oil palm based materials from palm oil milling and palm oil mill effluent (POME) as a source of bioactive compounds.

Present invention claims priority over a Malaysian Palm Oil Board (MPOB) Malaysian patent application PI2012700505 wherein said patent discloses bioactive compound with molecular weight of 482. Present invention further discloses the identity of said compound and characterizing the chemical structure of said compound.

Further objects and advantages of the present invention may become apparent upon referring to the preferred embodiments of the present invention as shown in the accompanying drawings and as described in the following description.

### SUMMARY OF INVENTION

In one aspect there is provided a composition comprising a compound obtained from oil palm based materials, wherein said compound is an indolacetic acid derivative. The composition is useful for providing bioactive properties. The compound having at least one variation of moiety, whereby the moiety is from but not limited to hydroxyl group and amide group. The moieties in the compound are preferably hydroxide molecule (—OH) and amidogen molecule (—NH₂). The composition in present invention comprises two variations of moiety in bioactive compounds in the ratio of 1:1.

Present invention also discloses a method of isolating bioactive compound obtained from oil palm based material comprising the steps of extracting said oil palm based material for oil palm phenolics (OPP), preparing compound for HPLC analysis, subjecting compound to HPLC analysis, isolating compound for fraction of interest, subjecting fraction to analytical HPLC analysis, quantifying and confirming the identity of fraction of interest, subjecting fraction to MALDI-TOF, and subjecting fraction to NMR analysis.

### BRIEF DESCRIPTION OF DRAWINGS

This invention will be described by way of non-limiting embodiments of the present invention, with reference to the accompanying drawing, in which:

FIG. 1 shows HPLC chromatogram of CSA and compound with peak 482;

FIGS. 2 show HPLC chromatogram of fractions isolated from oil palm phenolics (OPP);

FIG. 3 shows HPLC chromatogram of isolated fraction 21;

FIG. 4 shows HPLC chromatogram of isolated fraction 22;

FIG. 5 shows HPLC chromatogram of MPOB peak 1;

FIG. 6 shows HPLC chromatogram of isolated peak 482 in between fraction 21 and 22;

FIG. 7 shows analytical HPLC chromatogram of MPOB peak 1;

FIGS. 8a and 8b show analytical HPLC chromatogram and spectrum of MYB (peak 482) respectively;

FIGS. 8c and 8d show analytical HPLC chromatogram and spectrum of MYC (3-CSA) respectively;

FIG. 9 shows MALDI spectrum of isolated peak 482;

FIG. 10 shows selected HMBC correlations of compound A; and

FIG. 11 shows chemical structure of compound in peak 482.

### DETAILED DESCRIPTION OF THE PREFERRED EMBODIMENTS

The present invention will now be described in detail with reference to the accompanying drawings.

Present invention discloses description and examples directed to a bioactive compound, composition and method thereof, whereby the bioactive compound with molecular weight of 482 is extracted from oil palm and/or oil palm-based materials.

The biologically active extracts of palm vegetation liquor useful in this invention are those obtained from the vegetation liquor of the palm oil milling process according to various conventional suitable means and processes.

Although the extract may contain a variety of compounds including phenolic compounds, fruit acids, fruit sugars and glycerol, starch, cellulose and hemicellulose, for purposes of standardization the concentrations of the extracts used were measured in terms of phenolic content i.e gailic acid equivalent.

Embodiments of the present invention are directed to a composition comprising a bioactive compound and other major phenolic compounds obtained from any part of the oil palm, oil palm based materials including vegetation liquor of palm oil processing and palm oil mill effluent (POME). The composition of the present invention can be prepared based on available or standard methods. It is expected that the preparation is safe and said composition is suitable for use in, but not limiting to, daily consumption including dietary supplements, nutraceuticals, and for health promoting purposes. It is further noted that the bioactive compound and its derivatives obtained based on the preferred embodiments of the present invention are suspected to exhibit antiviral and antimicrobial effects.

It would be apparent to a person skilled in the art that the raw extracts obtained from any part of the oil palm, or oil palm based materials, including the vegetation liquor from palm oil milling and palm oil mill effluent for the purpose of the present invention may contain various other phenolic compounds in addition to the novel bioactive compound.

The extracts obtained from oil palm based materials and more particularly for this disclosure, the vegetation liquor from palm oil milling and palm oil mill effluent when subjected to isolation and purification stages in accordance with the method of the present invention are found to contain a novel bioactive compound or their derivatives.

The present invention extends, therefore to a novel bioactive compound; and method thereof, whereby the primary steps of said method are pre-treatment of raw extracts obtained from any part of the oil palm, the vegetation liquor from palm oil milling and palm oil mill effluents. The LCMS analysis of OPP extracted has found that fraction 3-CSA shows two prominent peaks of 3-CSA and an unknown compound with molecular weight of 482. This embodiment encompasses characterization of substantially purified bioactive compound having molecular weight of 482.

It should be noted that an “isolated or purified” bioactive compound or biologically active portion thereof, is substantially free of other cellular materials or other components or substantially free of chemical precursors or other chemicals.

Generally, peak 482 is isolated by a two steps process of general prep HPLC to isolate fractions containing peak 482 and isolating peak 482 from the fractions. The purity and identity of isolated compound having peak 482 are confirmed by using analytical HPLC. To overcome the elution problems caused by sensitivity of compound with peak 482 towards pH of mobile phase, a novel procedure to quantify and confirm the identity of peak 482 in analytical HPLC is developed.

The method of isolation, purification and characterization of the present invention is further illustrated by the following experimental examples. It should be understood that these experimental examples, while indicating preferred embodiments of the invention, are given by way for better elucidation only. A person skilled in the art can ascertain the essential characteristics and embodiments of this invention, therefore various changes may be provided to adapt to various usages and conditions.

### EXAMPLE 1

### Preparation of the Compound

In order to avoid blockage and pressure up-build in column prior to compound injection, the bioactive compound is pre-treated to obtain pre-concentrated or partially purified extracts free from hemi-cellulose and cellulose materials. This may be performed with low stringent conditions of subjecting the extracts to a flash chromatography or the likes, or alternatively, subjecting said extracts to ethanol precipitation, prior to separation by high performance liquid chromatography. An example of such method is given by way of reference below and should not be construed as limiting the scope of the claims.

The main steps involved for the first approach is precipitating the two substances with approximately two volumes of cold ethanol before vortexing and allowing mixture to coalesce in refrigerator for one hour. The coalesced mixture is centrifuged for approximately 12 mins at about 2000 rpm before subjecting supernatant from said substances for evaporation and reconstituted 5× with water for HPLC analysis. The dried product was dissolved in predetermined amount of distilled water for use in HPLC analysis. It should be mentioned that these steps might be substituted with alternative steps of standard procedures known in the art to achieve a similar objective.

The next imperative step of the method for the preparation of the composition as disclosed involves the isolation and purification of the phenolic compound from the partially purified or pre-treated extracts. This can be carried out with the conventional high performance liquid chromatography (HPLC) based on low stringent conditions or parameters. An example of such method is given by way of reference below and should not be construed as limiting the scope of the claims.

### EXAMPLE 2

### Preparation of HPLC for Fractionation

HPLC is used to isolate compound of peak 482 from OPP. In one preferred embodiment, an Econosil C18 5 μm particle size, with the preferred column length of 25 cm×10 mm id, flow rate of 3 ml per minute was prepared. The preferred mobile phase gradient may comprises two solvents, with one solvent consisting of 0.1% trifluoroacetic acid (TFA) and another solvent consisting of 90/10 of acetonitrile (ACN) with 0.1% TFA. In this study, the injection column was 1 ml and readings were taken at 280 nm. Table 1 shows the HPLC procedure for fractionation of sample OPP in accordance to one's preferred embodiment.

TABLE 1
Column: Econosil C18 5 μm L 25 cm Id 10 mm
Flow rate: 3 ml/min
Mobile Phase Gradient

| | Solvent A | Solvent B |
|---|---|---|
| Time | 0.1% TFA | 90/10 ACN/0.1% TFA |
| 0 | 90 | 10 |
| 1 | 90 | 10 |
| 55 | 60 | 40 |
| 60 | 0 | 100 |
| 65 | 0 | 100 |
| 70 | 90 | 10 |

Injection volume: 1 ml
Detector 280 nm

In another preferred embodiment for isolation of peak 482, an Econosil C18 5 μm particle size, with the preferred column length of 25 cm×10 mm id, flow rate of 3 ml per minute was prepared. The preferred mobile phase gradient may comprises two solvents, with one solvent consisting of 0.1% trifluoroacetic acid (TFA) and another solvent consisting of 90/10 of acetonitrile (ACN) with TFA volume per volume (v/v). In this study, the injection column was 3 ml and readings were taken at 275 nm. Table 2 shows the HPLC procedure for isolation of peak 482 in accordance to one's preferred embodiment.

TABLE 2
Column: Econosil C18 5 μm L 25 cm Id 10 mm
Flow rate: 3 ml/min
Mobile Phase Gradient

| | Solvent A | Solvent B |
|---|---|---|
| Time | 0.1% TFA | 90/10 ACN/TFA |
| 0 | 85 | 15 |
| 2 | 85 | 15 |
| 30 | 82 | 18 |
| 35 | 0 | 100 |
| 40 | 85 | 15 |

Injection volume: 1 ml
Detector 280 nm

It would be understood that the choice of columns and parameters for HPLC may vary however to obtain a similar result of elution time as described. Eluted fraction may be suitably collected and provided in powder or liquid form for use in further analysis.

### EXAMPLE 3

### Isolation of Fractions

FIG. 2 shows the HPLC chromatogram of OPP ethanol extract, wherein the effluent was collected at predetermined time. For Fraction 21 (Fr21), effluent was collected at about 40 mins; Fraction 22 (Fr22) at about 44 mins, and Fraction 23 (Fr23) at about 46 mins. Peak 3-CSA was found to be contained in Fraction 23, and LC-MS chromatogram showed that peak 482 was eluted slightly before 3-CSA in Fr23. The collected solution for these fractions are then concentrated using freeze drier.

### EXAMPLE 4

### Isolation of Compound in Peak 482

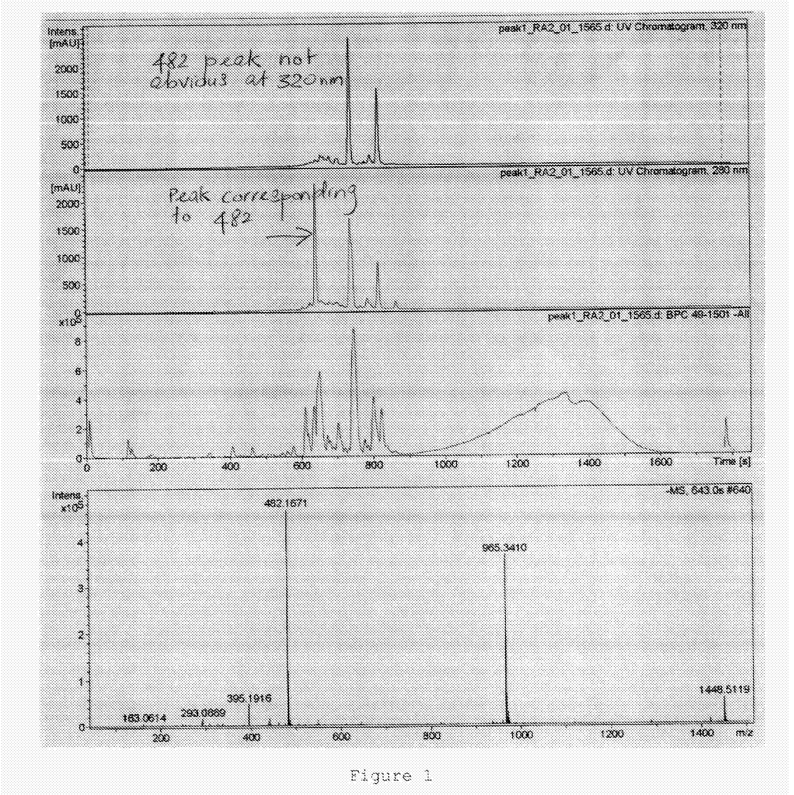

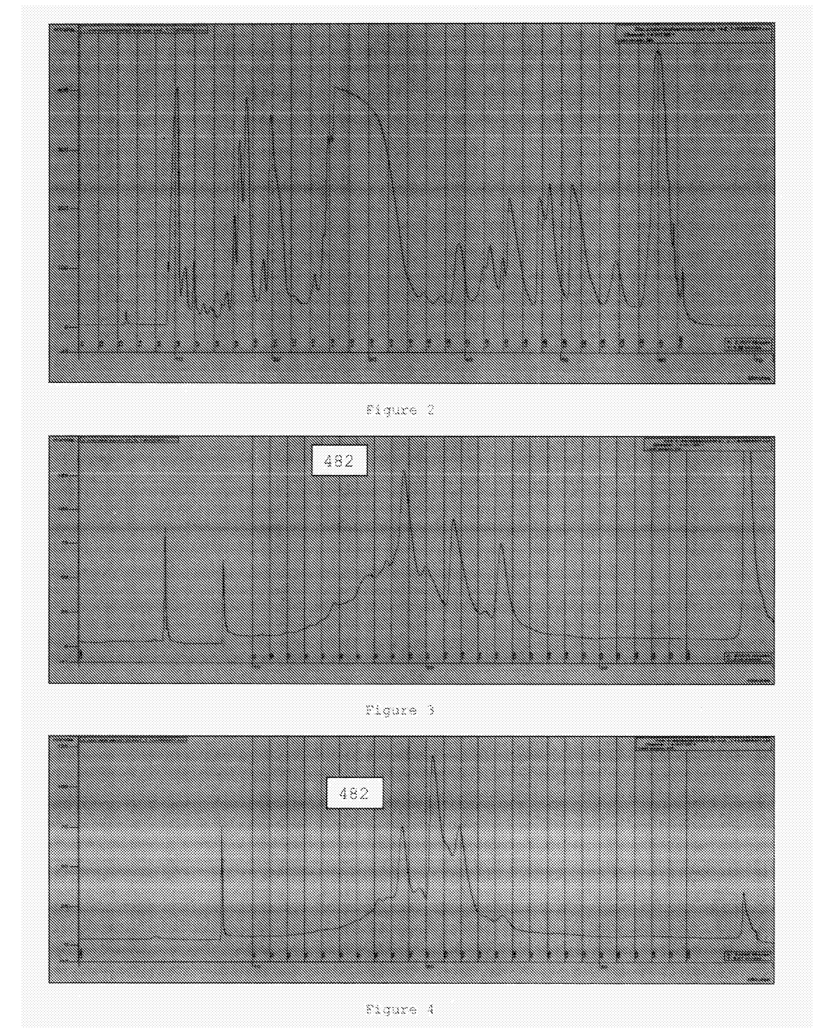

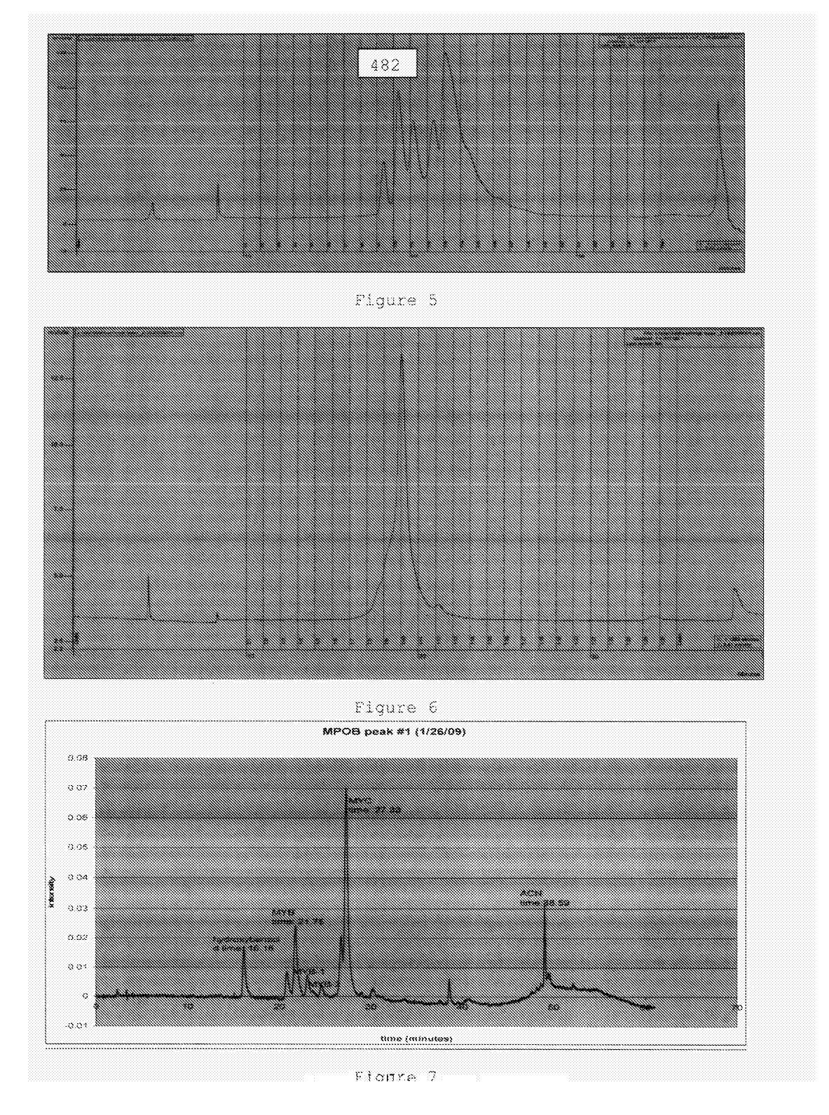

The lyophilized fractions were reconstituted in distilled water before injecting into prep HPLC to isolate compound in peak 482. FIGS. 3-5 show the HPLC chromatogram for Fr21, Fr22 and MPOB peak 1 respectively. Peak 482 is not contained in Fr2. In reference to FIG. 5, the chromatogram obtained for MPOB peak 1 sample is compared with peaks in Fr21 and Fr22 (FIGS. 3 and 4), the fraction in MPOB peak 1 was shown to contain compounds with mass of 483 as shown in FIG. 1. The fraction of peak 482 is collected and freeze dried. The compound was analyzed with prep HPLC again to ensure its purity before analyzing with NMR.

### EXAMPLE 5

### Analytical HPLC

Analytical HPLC is used to verify peak 482 as a distinct compound rather than some isomer of 3-CSA. In one preferred embodiment, a SGE Exsil ODS 5 μm particle size, with the preferred column length of 250 mm×4.6 mm GL, flow rate of 0.8 ml per minute was prepared. The preferred mobile phase gradient may comprises two solvents, with one solvent consisting of 0.02% H₃PO₄ and another solvent consisting of 50/50 of ACN/Methanol (MEOH) v/v. In this study, the injection column was 20 μl and readings were taken at 280 nm. Table 3 shows the HPLC procedure for fractionation of sample OPP in accordance to one's preferred embodiment.

TABLE 3
Column: SGE Exsil ODS 5 μm 250 × 4.6 mm GL
Flow rate: 0.8 ml/min
Mobile Phase Gradient

| | Solvent A | Solvent B |
|---|---|---|
| Time | 0.1% TFA | 90/10 ACN/0.1% TFA |
| 0 | 95 | 5 |
| 1 | 95 | 5 |
| 30 | 80 | 20 |
| 40 | 65 | 35 |
| 45 | 0 | 100 |
| 50 | 0 | 100 |
| 60 | 90 | 10 |

Injection volume: 20 μl
Detector 280 nm

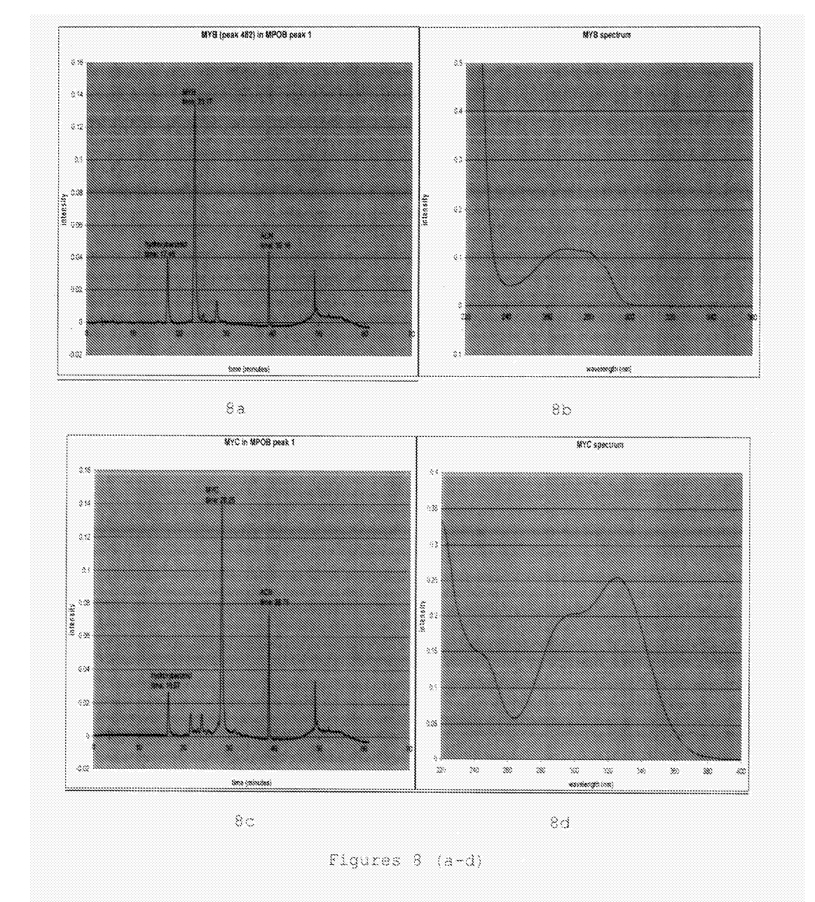

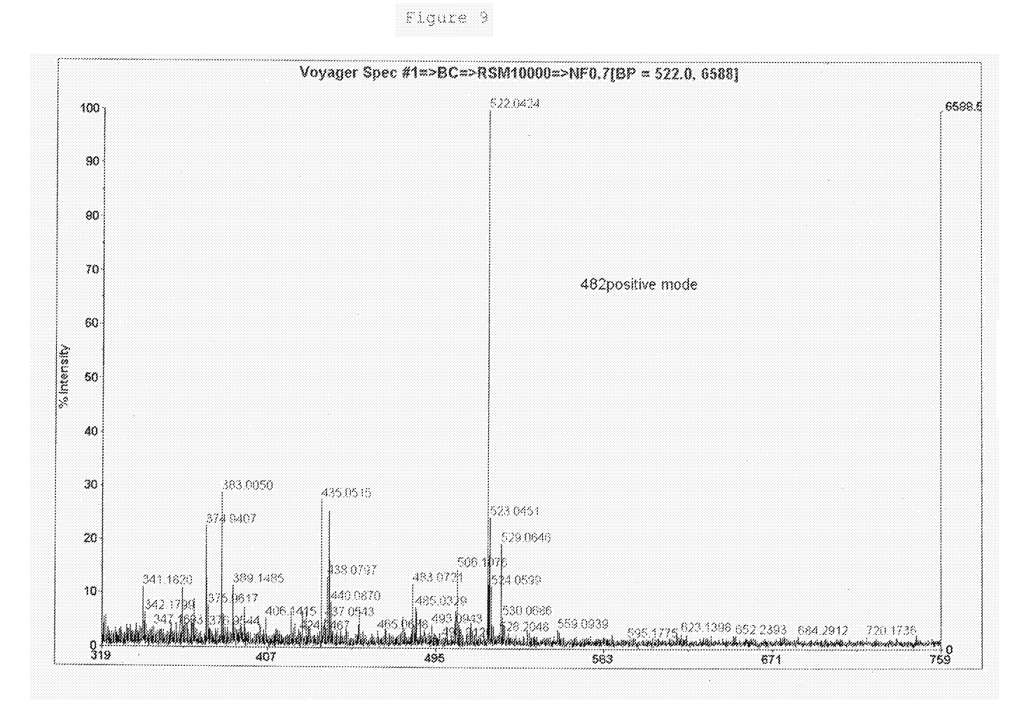

FIG. 7 shows the major peaks of MPOB peak 1 in analytical HPLC. In reference to FIGS. 7 and 8, MYB refers to peak 482 while MYC refers to 3-CSA. A pure sample of peak 482 (MYB) and a pure sample of 3-CSA (MYC) are run in analytical HPLC and compared their spectrum. As shown in FIGS. 8b and 8d, the two spectrums are distinct, hence it is verified that peak 482 is a unique compound.

### EXAMPLE 6

### MALDI-TOF—Mass Analysis

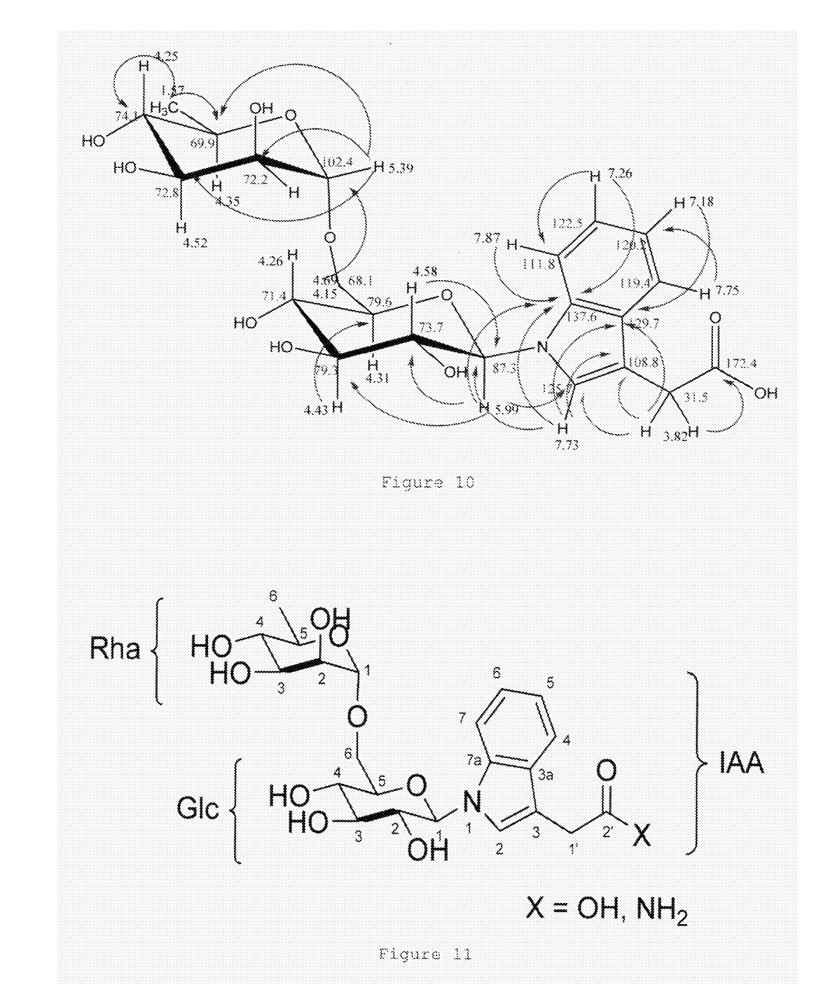

Each of the collected fractions were lyophilized to 5 μl, diluted with 5 μl of distilled water, ziptipped (c18) and eluted in 5 μl of 0.1 TFA, 70% ACN. The fractions are analysed in positive mode by spotting 1 μl of the fraction on plate without matrix solution before analysing. FIG. 9 shows the MALDI spectrum of isolated peak 482 wherein said fractions are ionized by (M+K)⁺.

### EXAMPLE 7

### NMR Analysis

In one's preferred embodiment, NMR spectra were recorded on a 3-channel Varian Inova spectrometer, operating at about 500 MHz for proton and about 125 MHz for carbon at 25° C. The probe used was a 5 mm, 3-channel, indirect detection and the solvent used was pyridine-d5. Proton and carbon chemical shifts were referenced to the residual solvent signals at 8.74 and 150.35 ppm correspondingly.

The compound in peak 482 consisted of an equimolar mixture of two compounds with the same hydrocarbon skeleton. Therefore, the structure elucidation and the proton and carbon chemical shifts assignment is shown in compound A (FIG. 10) are speculated to have the same correlations in the second compound.

### EXAMPLE 8

### The 3-indolyacetic Acid (IAA) Moiety

The sequence of four ortho-phenylene protons has been identified in the DQCOSY spectrum. The carbons carrying these protons have been assigned from the gHMQC spectrum. The quaternary carbons on the ortho-phenylene moiety have been identified in the gHMBC spectrum by couplings with the two protons ortho to them.

The gHMBC spectrum revealed long-range couplings between the protons and the carbons of an aromatic methine (7.73 on 125.7) and an aliphatic methylene (3.82 on 31.5). 7.73 does not couple with 3.82, therefore they are four bonds away. Both of 7.73 and 3.82 couple with 108.3 and 129.7, therefore one is the carbon between 125.7 and 31.5, and the other is attached to this carbon. 129.7 is already part of the benzene ring, and this assigns C2, C3 and C1′.

3.82 displays in the gHMBC spectrum one more long-range coupling, with 172.4, a chemical shift for a derivative of a carboxylic acid. Based on the lack of other couplings with carbons, and on the chemical shifts of 3.82 and 31.5, the carboxyl group was attached to 31.5. This was later confirmed by the molecular weight of the compounds.

7.73 couples with the other aromatic quaternary carbon, at 137.6. Carbon chemical shifts 111.8 and 108.8 suggested that a nitrogen lies between 127.5 and 137.6. The 3-indolyl acetic moiety was confirmed by the similarity of the chemical shifts with other prior arts and later by the molecular weight of the compounds.

### Example 9

### The β-glucopyranose (Glc) Moiety

C2 and C7a in IAA couple with a proton at 5.99 and H2 in IAA couples with the carbon at 87.3, caring 5.99, therefore 87.3 is attached to N1 in IAA. The sequence of protons in the Glc moiety was revealed by the DQCOSY spectrum, which also displayed large active couplings in the cross-peaks H1-H2, H2-H3, H3-H4 and H4-H5, therefore the stereochemistry is that of β-glucopyranose

### EXAMPLE 10

### The α-rhamnopyranose (Rha) Moiety

H6a and H6b in Glc couple with a carbon at 102.4; the proton on this carbon, 5.39, couples with C6 in Glc. The sequence of protons in the Rha moiety was identified in the DQCOSY spectrum. A TOCSY1D experiment with selective excitation of the methyl protons at 1.5 revealed two large (9-10 Hz) couplings for H4 at 4.25, therefore H3, H4 and H5 are all axial. A TOCSY1D experiment with selective excitation of H1 revealed that H2 has no couplings larger than 4 Hz, therefore it is equatorial. The stereochemistry of C1 was assigned to the alpha anomer, based on the 13C chemical shifts of C3 and C5.

### EXAMPLE 11

### The X Moiety

Therefore except for protons in the IAA moiety and surrounding the carboxyl group, H2, H4 and H1′, indicates that the two compounds differ in the type of carboxyl derivative. Preliminary MS data showed just one compound, MW=483, which is the weight of the carboxylic acid. The amide would be 482. The two compounds were assigned as carboxylic acid and amide based on the 13C chemical shifts of C2′.

It was concluded that RHA-482 is a 1:1 mixture of two very similar compounds. The structures are identified as in the FIG. 11, wherein X can be —OH and —NH₂.

TABLE 4

| | | X = OH | | X = NH₂ | |
|---|---|---|---|---|---|
| Moiety | Position | □H (ppm) | □C (ppm) | □H (ppm) | □C (ppm) |
| IAA | 2 | 7.73 | 125.7 | 7.82 | 125.6 |
| | 3 | — | 108.8 | — | 109.9 |
| | 3a | — | 129.7 | — | 129.4 |
| | 4 | 7.75 | 119.4 | 7.90 | 119.7 |
| | 5 | 7.18 | 120.2 | 7.17 | 120.1 |
| | 6 | 7.26 | 122.5 | 7.25 | 122.4 |
| | 7 | 7.87 | 111.8 | 7.87 | 111.8 |
| | 7a | — | 137.6 | — | 137.7 |
| | 1′ | 3.82 | 31.5 | 3.99 | 32.3 |
| | 2′ | nm^{a} | 172.4 | nm | 174.6 |
| Glc | 1 | 5.99 | 87.3 | 6.00 | 87.2 |
| | 2 | 4.58 | 73.7 | 4.60 | 73.7 |
| | 3 | 4.43 | 79.3 | 4.44 | 79.3 |
| | 4 | 4.26 | 71.4 | 4.26 | 71.4 |
| | 5 | 4.31 | 79.6 | 4.32 | 79.6 |
| | 6 | 4.69, | 68.1 | 4.69, | 68.1 |
| | | 4.15 | | 4.15 | |
| Rha | 1 | 5.39 | 102.4 | 5.39 | 102.4 |
| | 2 | 4.49 | 72.2 | 4.49 | 72.2 |
| | 3 | 4.52 | 72.8 | 4.52 | 72.8 |
| | 4 | 4.25 | 74.1 | 4.25 | 74.1 |
| | 5 | 4.35 | 69.9 | 4.35 | 69.9 |
| | 6 | 1.57 | 18.6 | 1.57 | 18.6 |

^{a}Not measured, due to exchange with the water in the solvent.

The novel compound of the claimed invention may be prepared for use in a pharmaceutically effective or nutraceutically effective amount, solely on its own or in combination with other agents or compounds deemed appropriate by a person skilled in the art. Further, compositions may be prepared in a manner, and in a form/amount as is conveniently practised.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A composition comprising a bioactive compound obtained from oil palm based materials, wherein said compound is an indolacetic acid derivative.

2. The composition as claimed in claim 1 wherein said compound is represented by formula I:

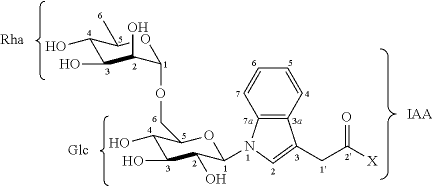

3. The composition as claimed in claim 2 wherein said compound having at least one variation of moiety (X).

4. The composition as claimed in claim 3 wherein said moiety is from a hydroxyl group.

5. The composition as claimed in claim 3 wherein said moiety is a hydroxide molecule (—OH).

6. The composition as claimed in claim 3 wherein said moiety is from an amide group.

7. The composition as claimed in claim 3 wherein said moiety is an amidogen molecule (—NH₂).

8. The composition as claimed in claim 3 wherein said compound comprising two variations of moiety in bioactive compounds in the ratio of 1:1.

9. The composition as claimed in claim 1 wherein said bioactive compound is extracted from palm oil based wastes.

10. The composition as claimed in claim 1 wherein said bioactive compound is extracted from oil palm vegetation liquor.

11. The composition as claimed in claim 1 wherein said bioactive compound is extracted from palm oil mill effluent.

12. The composition as claimed in claim 1 is useful for providing bioactive properties.

13. A method of isolating bioactive compound obtained from oil palm based material comprising the steps of:
extracting said oil palm based material for oil palm phenolics (OPP),
preparing compound for HPLC analysis,
subjecting compound to HPLC analysis, and
isolating compound for fraction of interest.

14. The method of isolating bioactive compound as claimed in claim 13 further comprising the step of subjecting said fraction to analytical HPLC analysis.

15. The method of isolating bioactive compound as claimed in claim 14 wherein said step of subjecting said fraction to analytical HPLC analysis further comprising the step of quantifying and confirming the identity of fraction of interest.

16. The method of isolating bioactive compound as claimed in claim 13 further comprising the step of subjecting said fraction to MALDI-TOF.

17. The method of isolating bioactive compound as claimed in claim 13 further comprising the step of subjecting said fraction to NMR analysis.
